(19) United States
(12) Patent Application Publication
(10) Pub. No.: US 2006/0068426 A1
(43) Pub. Date: Mar. 30, 2006

(54) Cyclic peptides and antibodies thereof

(76) Inventors: James P. Tam, Palm Beach, FL (US); Chadler Pool, Phoenixville, PA (US); Ying Zhang, Nashville, TN (US); Kristen Sadler, Singapore (SG)

Correspondence Address: FULBRIGHT & JAWORSKI L.L.P., 600 CONGRESS AVE., SUITE 2400, AUSTIN, TX, 78701, US

(21) Appl. No.: 11/207,573

(22) Filed: Aug. 19, 2005

Related U.S. Application Data
(60) Provisional application No. 60/603,449, filed on Aug. 20, 2004.

(51) Int. Cl. C12Q 1/70; C12Q 1/68

(52) U.S. Cl. 435/6; 435/5

(57) ABSTRACT

The present invention provides methods of preparing and identifying antibodies against a loop domain of a protein, such as an extracellular loop (ECL) domain of a transmembrane protein. Cyclic and end-to-end cyclized peptides corresponding to loop domains are employed in the present invention. Transmembrane proteins contemplated by the invention include the G-coupled protein receptor or a viral envelope protein.

This application claims benefit of priority to U.S. Provisional Application Ser. No. 60/603,449, filed Aug. 20, 2004, the entire content of which is hereby incorporated by reference.

The government owns rights in the present invention pursuant to grant number NIH A146164 from the National Institutes of Health.

### BACKGROUND OF THE INVENTION

1. Field of the Invention

The present invention relates generally to the fields of molecular biology, immunology, and immunotherapy. More particularly, it concerns methods of preparing and identifying antibodies against cyclic peptides corresponding to the extracellular loop (ECL) domain of a transmembrane protein.

2. Description of Related Art

The concept that the more closely antigens resemble native structure the greater the chance of producing antibodies of appropriate quality and quantity is certainly not new. It is well-documented that the use of whole proteins as antigens generally results in the production of antibodies capable of recognizing and binding to native targets. However, protein antigens often induce a complex mixture of polyclonal antibodies with varying specificities, and unless the target epitope is immunodominant, specific antibody quantity may be inadequate. This case is particularly acute in producing effective anti-HIV antibodies where critical target epitopes are protected by larger immunodominant variable regions and whole proteins representing the native structure are unsuitable as antigens (McMichael and Hanke, 2003; Veljkovic et al., 2001). Peptide antigens are now frequently used to induce antibodies with correct specificity. However, data supporting their ability to mimic structural epitopes, both sequential and discontinuous, is not comprehensive. This is due mainly to the fact that it is usually more challenging to mimic epitopes with a high degree of structural integrity using relatively short peptides. One approach to overcome this drawback is the formation of constrained peptides.

### SUMMARY OF THE INVENTION

The present invention overcomes deficiencies in the prior art by providing highly active, well-characterized antibodies against protein loop domain, and more particularly a transmembrane loop domain (extracellular loop (ECL) or intracellular loop (ICL)). In a particular embodiment, the present invention provides a method for preparing an antibody against a protein loop domain comprising (a) providing a cyclic peptide corresponding to the loop domain; (b) administering the cyclic peptide with an experimental animal under conditions supporting production of antibodies; and (c) obtaining from the animal (i) an antibody that binds to the loop domain or (ii) an antibody-producing cell, antibodies from which bind to the loop domain. In further embodiments, the invention comprises the steps of fusing an antibody-producing cell with an immortalized cell. The cyclic peptide may be linked to a carrier molecule. In still another embodiment, the cyclic peptide of step (b) may be mixed with an adjuvant. In another embodiment, the present invention further comprises assessing binding of the antibody with the cyclic peptide. The cyclic peptide may comprise multiple loop domains from a single protein, i.e., transmembrane protein, and may include both ECL and ICL domains. One may also use various linkages to connect multiple cyclic peptides together, each reflecting a unique protein loop domain (e.g., ECL-ECL . . . , ICL-ICL . . . , or ECL-ICL . . . ).

In other embodiments, the present invention further comprises assessing binding of the antibody with native protein, e.g., a transmembrane protein. The transmembrane protein may be a G-protein coupled receptor such as CCR5, CXCR4, CR7 and CXCR3. In other embodiments of the invention, the transmembrane protein may be a viral envelope protein. A viral envelope protein as contemplated in the present invention may include, but is not limited to, a human rhinovirus protein, influenza A virus protein, sendai virus protein, Herpes simplex virus (type 1) protein, Epstein-Barr virus protein, vesicular stomatitis virus protein, rabies virus protein or a human immunodeficiency virus protein.

An experimental animal as contemplated by the present invention may be, but is not limited to, a mouse, a rat, a rabbit, a guinea pig, a goat, a sheep, a non-human primate or a human.

In another particular embodiment, the present invention provides a method for identifying an antibody against a protein loop domain comprising (a) providing a cyclic peptide corresponding to the loop domain; (b) contacting the cyclic peptide with a population of antibodies; and (c) identifying an antibody that binds to the protein domain.

As contemplated by the present invention, the population of antibodies may be expressed on the surface of a phage library or may be comprised in an antibody array. In other embodiments, the present invention further comprises assessing binding of the antibody with a corresponding native protein.

In yet another embodiment, the present invention further comprises assessing binding of the antibody with the cyclic peptide. In some embodiments of the present invention, the peptide may be labeled and mixed in solution with the phage library.

In still yet another particular embodiment, the present invention provides an end-to-end cyclized peptide corresponding to a protein loop domain. Such peptide may further comprise a heterologous thiol residue. In some embodiments of the invention the peptide may be labeled with a colorimetric label, a chemilluminescent label, a fluorimetric label, a radiolabel, a dye or a ligand such as biotin. In still yet a further embodiment, the cyclized peptide comprises an amide bond that fuses both ends.

In further embodiments, the protein to which the end-to-end cyclized peptide corresponds may be a transmembrane protein, for example, an ECL or ICL domain thereof, such as from a G-protein coupled receptor, such as, CCR5, CXCR4, CR7 and CXCR3. In still further embodiments, the transmembrane protein may be a viral envelope protein, such as a human rhinovirus protein, influenza A virus protein, sendai virus protein, Herpes simplex virus (type 1) protein, Epstein-Barr virus protein, vesicular stomatitis virus protein, rabies virus protein or a human immunodeficiency virus protein.

Embodiments discussed with respect to one embodiment or example of the invention may be employed or implemented with respect to any other embodiment of the invention.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C. Schematic representations of CCR5. FIG. 1A—TM helical segments represented by cylinders are held in a complex conformational arrangement by intra- and extracellular domains and two disulfide bonds. The extracellular loops (bold) and Cys residues (o) are indicated. FIG. 1B—Structure of CCR5 with residues from ECL1, ECL2 and ECL3 selected for peptide synthesis shown in grey. FIG. 1C—Primary structures of the six peptides designed to mimic the extracellular domains of CCR5. Underlined residues were added to peptides destined for cyclization to facilitate reaction and act as spacers. Italicized residues are from TM segments which support the extracellular loops.

FIGS. 2A-2B. Design of molecules used for phase selection. Phage specific for test peptides were selected by binding to biotinylated peptides, biotinylated ELC is shown with ECL2 residues highlighted grey (FIG. 2A). The linker peptide (FIG. 2B) was included to remove phage specific for the non-CCR5 residues.

FIG. 3. The selection strategy developed for capturing phase expressing ScFv antibodies specific for peptides mimicking CCR5 structural epitopes. Biotinylated peptides were incubated in solution with a phage library before addition to streptavidin-coated beads. Captured phage were eluted and amplified before crude ScFv collection and screening of the ScFv antibodies for binding to CCR5+ cells. Single-chain antibodies that exhibited the strongest binding to CCR5+ cells were purified and used in subsequent assays.

FIGS. 4A-4C. Binding of ScFv antibodies to CCR5+ cells. FIG. 4A—Specific binding of ScFv antibody to CCR5 shown by typical Western blot profile. Cell lysates from Cf2Th/syn CCR5 cells (Lane 1) and PM 1 cells (Lane 2) probed with ScFv N18. FIG. 4B—Sup-T1 cells were either incubated alone (black), or with ScFv A1 (red) or mAb 2D7 (green) then stained with FITC conjugate and a CCR5+ sub-population examined by flow cytometry. FIG. 4C—Indirect immunofluorescent staining of CCR5+ cells with ScFv antibody A1. Cf2Th/syn CCR5 cells were incubated with ScFv antibody and binding visualized by addition of anti-E-tag FITC conjugate (magnification ×100).

FIG. 5. Inhibitory activity of selected ScFv antibodies against M-tropic HIV BaL. P4R5 cells expressing CXCR4 and CCR5 were used to measure ScFv biological activity. Cells were treated with ScFv then infected with CCR5-tropic HIV-1 BaL. The number of resulting infectious centers per well is expressed as a percentage of the number in the control wells that did not receive antibody. Control wells either received MAb 2D7 in place of ScFv or no antibody. An admix of the three ScFv antibodies N4, O17 and P21 was also tested and denoted “Admix”. Arrows indicate ScFv that displayed significant inhibitory activity.

### DESCRIPTION OF ILLUSTRATIVE EMBODIMENTS

Peptides have been utilized extensively to produce antibodies against discrete regions of larger polypeptides. However, a continuing problem with the use of peptides as antigens or immunogens is the poor quality of results being generated. Peptides are fragmented proteins and fairly flexible without constraints. As a result, they usually can not mimic the structure of the proteins from which they are derived. Thus, relatively, peptides are poor substitutes for proteins which are held rigidly together by the folded structures.

One approach to mimic a contiguous bioactive surface of a protein is to constrain the peptide chains into cyclic structures. This has been successfully validated in the development of many small peptides as receptor agonists or antagonists, enzyme substrates or inhibitors, or antigens. Constrained peptides usually have defined conformations in aqueous solution that may mimic the bioactive conformations. Recent studies using phage-displayed cyclic peptides in the development of peptide agonists for cytokines such as erythropoietin and TNF have further validated the importance of constraining peptides in obtaining good-quality bioactive peptides. However, to date, there are limited examples of cyclic peptides as antigens.

### I. THE PRESENT INVENTION

Thus, the present invention relates to the generation of antibodies to cyclic peptides corresponding to the protein loop domains, and in particular, the extracellular loop (ECL) domain of a transmembrane protein and/or an end-to-end cyclized peptide corresponding. Such antibodies are useful as immunotherapeutics in treating or preventing a disease or condition, or diagnostic reagents therefor.

This approach has particular application in the design of antigens that are anticipated to closely resemble natural CCR5 domains. As well as serving as useful tools for studying the functional properties of CCR5 in vitro and in vivo, it is envisioned that such single chain antibodies will serve as effective HIV inhibitors (Tsamis et al., 2003; Blanpain et al., 2002). The CCR5 and CXCR4 chemokine receptors serve as the major coreceptors on macrophages and T lymphocytes for human immunodeficiency virus type 1 (HIV-1) isolates, termed M- and T-tropic isolates, respectively (Moore et al., 1997; Deng et al., 1996). Although various CCR5-targeted molecules, including small chemicals, peptides and proteins including antibodies, have been investigated for inhibitory capacity (Cackerian et al., 1999; Zuber et al., 2000; Steinberger et al., 2000; Osbourn et al., 1998; Tsamis et al., 2003; Kazmierski et al., 2003; Wu et al., 1997; Olson et al., 1999) and the antibody production approach has been successful, it requires a painstaking effort to isolate active antibodies from the complex mixtures of irrelevant antibodies. Furthermore, even though progress in isolation of anti-CCR5 antibodies has been steady, there are still very few highly active, well-characterized monoclonal antibodies (MAbs) available against the extracellular domains of CCR5, particularly against the ectodomain loops, ECL1 and ECL3. Thus, there is a need in the art for antibodies against the extracellular loop (ECL) domain of proteins i.e., transmembrane (TM) proteins that can be useful in the identification of immunotherapeutics.

Cyclic peptides and corresponding linear peptides derived from the three extracellular domains of CCR5 were synthesized and used as antigens to select recombinant single-chain antibodies (ScFv) from a constructed phage display library. Using peptides for two rounds of solution-phase phage selection and CCR5+ cells for assays (Tsamis et al., 2003), ScFv antibodies that bound to CCR5+ cells were isolated by flow cytometric analysis. Three of the single-chain antibodies exhibited the ability to inhibit infection by M-tropic HIV-1 BaL. The results demonstrate that peptides designed to closely mimic structural epitopes of CCR5 are effective tools for the isolation of biologically active site-specific ScFv antibodies. This approach provides useful reagents for structure-function analysis of the role of CCR5 in HIV-1 infection and holds promise for developing immunotherapeutics as pre-entry inhibitors.

Thus, the present invention demonstrates that cyclic peptides representing the extracellular domains of a receptor are more likely to mimic native CCR5 than linear peptides, and that single chain antibodies selected using a novel selection strategy inhibit a relevant biological function. Thus, this study provides proof of principal for the design of peptide antigens generally. Since TM receptors with ECL are abundantly represented in membrane-anchored proteins, the strategy of using loop-mimicking antigens provides for the possibility of rapidly generating antibodies against discrete epitopes on these TM-containing proteins.

In cases where secondary, tertiary and quaternary structural data is inadequate or not available, sequence analysis is the tool most often used to identify potential domains, such as extracellular loops or transmembrane segments, within proteins. The amino acid sequences and associated characteristics e.g., hydrophobicity and secondary structure potential, of proteins with unknown structure are analyzed for homology with defined proteins. High homology between sequences allows an estimation of domain positions within a sequence.

### II. TRANSMEMBRANE PROTEINS AND PEPTIDES

In certain embodiments, the present invention concerns compositions comprising at least one cyclic peptide corresponding to an ECL domain of a transmembrane protein, including an end-to-end cyclized peptide thereof. Cyclic peptides, due to their constrained conformation, provide enhanced stability and specific activity than linear peptides. Cyclic peptides of the present invention are of tremendous benefit in the design of antigens and in antibody selection.

Transmembrane proteins are well known in the art and generally refer to as integral membrane proteins that span the internal and external surfaces of the biological membrane or the phospholipid bilayer in which it is embedded. Transmembrane proteins may be grouped into single pass or multiple pass proteins (i.e., the number of times they span the lipid bilayer). Such proteins may include, for example, G-protein coupled receptor such as CXCR4, CR7, CCR5 and CXCR3; cell adhesion proteins such as selectins, integrins and cadherins; and insulin receptor, but are not limited to such.

Transmembrane protein may comprise one or more extracellular domains. The extracellular domain refers to the portion of the receptor that sticks out of the membrane on the outside of the cell or organelle. If the polypeptide chain of the receptor crosses the bilayer several times, the external domain can comprise several “loops” sticking out of the membrane. As used herein, the terms “extracellular domain or extracellular loop domain” may be used interchangeably. In preferred embodiments of the invention, the ECL domains of transmembrane proteins such as the G-protein coupled receptor and viral envelope proteins are employed. In particular embodiments, the extracellular domains of the G-protein coupled receptor, CCR5, are represented herein by SEQ ID NO:1, SEQ ID NO:2, SEQ ID NO:3, SEQ ID NO:4, SEQ ID NO:5, and SEQ ID NO:6.

It is contemplated that one may incorporate, into a single cyclic peptide, multiple loop domains. The loop domains may be reflective of multiple loop domains within a single protein, such as a transmembrane protein, and may also include multiple ECLs, ICLs, or a mix of ECLs and ICLs. To effect a similar structure, multiple cyclic peptides maybe linked through covalent bonds to create a plurality of ECLs, ICLs or a mixture of ECLs and ICLs.

The term “peptide” as used herein refers to the inventive cyclic peptide corresponding to an protein loop domain, such as an ICL or ECL domain of a transmembrane protein, including an end-to-end cyclized peptide corresponding thereto. As used herein, cyclic peptides corresponding to a loop domain refer to a relatively short peptide sequence. Typically, these peptides will be between about 7 amino acids to about 100 amino acids in length, however any peptide of any length that is useful and that is within the scope of this definition provided herein is considered. Specifically, such peptides maybe 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, or more amino acid residues in length. Further, the peptides may be described in terms of any range of lengths derivable between the integers disclosed above, for example, but not limited to lengths between 10-15, 10-25, 12-30, and 20-40 amino acids or greater lengths. Peptides as contemplated in the present invention may include any amino acid sequence containing a known or putative extracellular or intracellular domain of an integral membrane protein. The extracellular or intracellular domain can be defined by mutational analysis, algorithms or other modes of analysis which are regularly used by those skilled in the art.

As used herein, an “amino acid or amino acid residue” refers to any naturally occurring amino acid, any amino acid derivative or any amino acid mimic known in the art, including modified or unusual amino acids such as but not limited to, 2-Aminoadipic acid; 4-Aminobutyric acid, piperidinic acid; 2-Aminopimelic acid; 2,4-Diaminobutyric acid; Desmosine; 2,3-Diaminopropionic acid; N-Ethylasparagine; Hydroxylysine; 3-Hydroxyproline; 4-Hydroxyproline; or Norleucine. The residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties.

It also will be understood that amino acid may include additional residues, such as additional N— or C-terminal amino acids, and yet still be essentially as set forth in one of the sequences disclosed herein, so long as the sequence meets the criteria set forth above, including the maintenance of biological activity.

It is also contemplated in the present invention that the cyclic peptide corresponding to an ECL domain of a transmembrane protein may comprise amino acid residues that have been replaced or substituted or inserted or added. Such amino acid modification may facilitate cyclization or may act as spacers. In other instances, such amino acid modifications may facilitate interaction with fusion proteins or protein linkers.

The following is a discussion based upon changing the amino acids of a protein or peptide, to create a modified protein or peptide. For example, certain amino acids may be substituted for other amino acids in the loop domain of a protein, resulting in a greater binding specificity, cell uptake and enhancement of an immune response. Since it is the interactive capacity and nature of a protein that defines that protein's biological functional activity, certain amino acid substitutions can be made in a protein sequence, and in its underlying DNA coding sequence, thereby producing a modified protein.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that for amino acids positioned in the homologous region of nucleotide and encodes for the protein or polypeptide, the substitution of pairs of homologous and non-homologous amino acids can be made effectively on the basis of polarity. Non-homologous amino acids may be conservatively substituted with a member of the same polarity group as defined below: basic amino acids: arginine (+3.0), lysine (+3.0), and histidine (-0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified peptide or protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The term “a sequence essentially as set forth in SEQ ID NO:1, SEQ ID NO:2, SEQ ID NO:3, SEQ ID NO:4, SEQ ID NO:5 or SEQ ID NO:6” or “a sequence essentially as set forth in SEQ ID NO:1, SEQ ID NO:2, SEQ ID NO:3, SEQ ID NO:4, SEQ ID NO:5 or SEQ ID NO:6” means that the sequence substantially corresponds to a portion of SEQ ID NO:1, SEQ ID NO:2, SEQ ID NO:3, SEQ ID NO:4, SEQ ID NO:5 or SEQ ID NO:6 and has relatively few amino acids that are not identical to, or biologically functionally equivalent to, the amino acids of SEQ ID NO:1, SEQ ID NO:2, SEQ ID NO:3, SEQ ID NO:4, SEQ ID NO:5 or SEQ ID NO:6. It is contemplated that embodiments discussed with respect to a SEQ ID NO may be applied or implemented with respect to any other SEQ ID NO described herein.

A protein or peptide of the present invention may comprise at least one of 20 amino acids in naturally synthesized proteins, or at least one modified or unusual amino acid as is known in the art. The term “functionally equivalent codon” is used herein to refer to codons that encode the same amino acid, such as the six codons for arginine or serine, and also refers to codons that encode biologically equivalent amino acids. Codons preferred for use in humans, are well known to those of skill in the art (Wada et al., 1990). Codon preferences for other organisms also are well known to those of skill in the art (Wada et al., 1990, included herein in its entirety by reference).

TABLE 1

| | |
|---|---|
| Amino Acid | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

A. Synthesis of Cyclic Peptides

Proteins or peptides may be made by any technique known to those of skill in the art, including through standard molecular biological techniques, the isolation of proteins or peptides from natural sources, or the chemical synthesis of proteins or peptides.

In particular embodiments, the present invention comprises constrained peptides. To prepare constrained peptides, linear peptide sequences need to be circularized. Many methods have been developed based on the conventional peptide synthetic schemes of using multiple-tier protection groups. For example, conventional cyclization to form cyclic peptides requires a protection scheme for selected functional side chains. The reactions are carried out in organic solvent (off resin) or on solid phase (on-resin). After cyclization, the side chain protecting groups are removed. However, these methods are not practical for generating constrained peptide arrays intended for screening phage-displayed antibodies.

Thus, the present invention provides two new and efficient methods to generate cyclic peptides using on-resin cyclization and released them in aqueous solutions that are suitable for direct assays. The first method utilizes the facile thiolactone formation between a side-chain thiol group and the C-terminal thioester. The thiolactone formation represents a sidechain-to-end constraint. It is accomplished by assembling a desired peptide sequence on a thioester resin, deblocking the side chain protecting groups and then cyclizing the peptide in aqueous conditions buffered at pH 7.5. The cyclization also simultaneously releases the peptide from the resin support. The cyclic peptide can then be used for assays. The second method utilized the same chemistry as the first to yield an end-to-end cyclic peptide. This is accomplished by placing the Cys at the N-terminus. Thiolactone formation effects cyclization and release of the peptide from the resin support as described earlier. The N-terminal thiolactone undergoes an S,N-acyl migration to spontaneously yield an end-to-end cyclic peptide.

An advantage of these two proposed methods is that they are complementary to each other both operationally and by design. Operationally, a Cys-scan of a given sequence yields the sidechain-to-end cyclic peptides while a truncated Cys-scan affords the end-to-end cyclic peptides. Furthermore, these two constrained shaped peptides represent a wide sampling of possible bioactive conformations than by a single constraining method. Included in these methods is the potential addition of amino acid residues at the N— and/or C-termini of the cyclic peptides to provide spacing at the site of conjugation as to allow the peptides to better mimic the conformation of an ECL. The number of additional residues can depend on the spacing of the transmembrane helices of a given protein and therefore can vary. Also, the additional residues can include, but are not limited to, lysine, arginine, glutamate, aspartate, histidine, or other residues that impart improved solubility to the cyclic peptide.

These methods meet the demand of speed and efficiency for generating peptides for array format. The conventional approaches requiring a scheme of multi-tiered protecting groups is usually too cumbersome while the new methods employing unprotected constrained peptides generated from their linear unprotected peptides in aqueous conditions is more efficient. These methods that use protecting groups to produce cyclic peptides directly for a variety of purposes, including antigen scanning and generation of constrained peptide libraries are of value in generating antibodies through the phage-displayed antibody libraries.

It is further contemplated by the present invention, that cyclic or end-to-end cyclized peptides corresponding to the loop domain of a protein may encompass biologically functional equivalent modified polypeptides and peptides through site-directed or site-specific mutagenesis of the underlying DNA. Techniques for site-directed mutagenesis are known to those of ordinary skill in the art, and can be practiced without undue experimentation in the context of the present invention.

Fusion proteins may also be employed in the present invention and may generally have all or a substantial portion of the native molecule, linked at the N— or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions may include linking of functional domains, such as active sites from enzymes such as a hydrolase, glycosylation domains, cellular targeting signals or additional transmembrane regions. Still other fusion protein may include phage display.

In addition to cyclic or end-to-end cyclized peptides, fusion proteins of the present invention may include virtually any protein or peptide that could be incorporated into a fusion protein comprising a cyclic or end-to-end cyclized peptide. Methods of generating fusion proteins are well known to those of skill in the art. For example, fusion proteins may be made by de novo synthesis of the complete fusion protein or by attachment of a cyclic peptide followed by expression of the intact fusion protein.

B. Protein Purification

It may be desirable to purify protein or peptide of the present invention such as, for example, a peptide corresponding to an ECL domain of a transmembrane protein of the present invention. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; or isoelectric focusing. A particularly efficient method of purifying peptides is fast protein liquid chromatography or even HPLC.

Certain aspects of the present invention concern the purification, and in particular embodiments, the substantial purification, of a peptide or protein. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will also be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (e.g., alter pH, ionic strength, and temperature).

In affinity chromatography, the matrix should be a substance that itself does not adsorb molecules to any significant extent and that has a broad range of chemical, physical and thermal stability. The ligand should be coupled in such a way as to not affect its binding properties. The ligand also should provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed herein.

### III. ANTIBODIES

A. Antibody Generation

In particular embodiments, the present invention provides methods of obtaining, preparing, and/or identifying an antibody against a protein domain. In particular, an ICL domain or an ECL domain of a transmembrane protein such as G-protein coupled receptor and related proteins such as CCR5, CXCR4, CR7 and CXCR3 is contemplated in the present invention.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is also used to refer to any antibody-like molecule that has an antigen binding region, and includes antibody fragments such as Fab′, Fab, F(ab′)₂, single domain antibodies, Fv, scFv (single chain Fv), and the like. The techniques for preparing and using various antibody-based constructs and fragments are well known in the art. Means for preparing and characterizing antibodies are also well known in the art (see, e.g., Harlow and Lane, 1988; incorporated herein by reference).

Antibodies to a cyclic peptide corresponding to an ECL domain of a transmembrane protein or a peptides such as a end-to-end cyclic peptide corresponding to the ECL domain of a transmembrane protein can be generated using standard techniques.

1. Methods of Generating Polyclonal Antibodies

The methods for generating polyclonal antibodies are well known to those of ordinary skill in the art (see Harlow et al., 2000). Briefly, a polyclonal antibody is prepared by immunizing an animal with an immunogenic composition in accordance with the present invention and collecting antisera from that immunized animal. A wide range of animal species can be used for the production of antisera. Typically the animal used for production of anti-antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant. Suitable molecule adjuvants include all acceptable immunostimulatory compounds, such as cytokines, toxins or synthetic compositions. Adjuvants that may also be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MHC antigens may even be used.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an ear vein or alternatively by cardiac puncture. The procured blood is allowed to coagulate and then centrifuged to separate serum components from whole cells and blood clots. The serum may be used as is for various applications or else the desired antibody fraction may be purified by well known methods, such as affinity chromatography using another antibody or a peptide bound to a solid matrix or protein A followed by antigen (peptide) affinity column for purification.

2. Methods of Generating Monoclonal Antibodies

MAbs may be readily prepared through use of well known techniques, such as those exemplified in U.S. Pat. No. 4,196,265, incorporated herein by reference, and Harlow et al. (2000). Typically, this technique involves immunizing a suitable animal with a selected immunogen composition, e.g., a purified or partially purified cyclic peptide corresponding to an ECL domain of a transmembrane protein or a end-to-end cyclized peptide or polypeptide corresponding thereto. The immunizing composition is administered in a manner effective to stimulate antibody-producing cells.

The methods for generating monoclonal antibodies (MAbs) generally begin along the same lines as those for preparing polyclonal antibodies. Rodents such as mice and rats are preferred animals, however, the use of rabbit, sheep, goat, monkey cells also is possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen, generally as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B cells), are selected for use in the MAb generating protocol. These cells may be obtained from biopsied spleens or lymph nodes. Spleen cells and lymph node cells are preferred, the former because they are a rich source of antibody-producing cells that are in the dividing plasmablast stage.

Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortalized cell, such as a myeloma cell, from preferably one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984; each incorporated herein by reference). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

3. Generating Hybrids of Antibody-Producing Cells

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide MAbs. The cell lines may be exploited for MAb production in two basic ways.

A sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration.

The individual cell lines could also be cultured in vitro, where the MAbs are naturally secreted into the culture medium from which they can be readily obtained in high concentrations.

MAbs produced by either means may be further purified, if desired, using filtration, centrifugation and various chromatographic methods such as HPLC or affinity chromatography. Fragments of the monoclonal antibodies of the invention can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate monoclonals. For this, combinatorial immunoglobulin phagemid libraries are prepared from RNA isolated from the spleen of the immunized animal, and phagemids expressing appropriate antibodies are selected by panning using cells expressing the antigen and control cells e.g., normal-versus-tumor cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Humanized monoclonal antibodies are antibodies of animal origin that have been modified using genetic engineering techniques to replace constant region and/or variable region framework sequences with human sequences, while retaining the original antigen specificity. Such antibodies are commonly derived from rodent antibodies with specificity against human antigens. Such antibodies are generally useful for in vivo therapeutic applications. This strategy reduces the host response to the foreign antibody and allows selection of the human effector functions.

The techniques for producing humanized immunoglobulins are well known to those of skill in the art. For example U.S. Pat. No. 5,693,762 discloses methods for producing, and compositions of, humanized immunoglobulins having one or more complementarity determining regions (CDR's). When combined into an intact antibody, the humanized immunoglobulins are substantially non-immunogenic in humans and retain substantially the same affinity as the donor immunoglobulin to the antigen, such as a protein or other compound containing an epitope.

Other U.S. Pat. Nos. 5,565,332, 4,816,567, and 4,867,973, each incorporated herein by reference, teach the production of antibodies useful in the present invention. For example, U.S. Pat. No. 5,565,332, describes the production of chimeric antibodies using a combinatorial approach; U.S. Pat. No. 4,816,567 describes recombinant immunoglobin preparations; and U.S. Pat. No. 4,867,973 describes antibody-therapeutic agent conjugates.

U.S. Pat. No.5,565,332 also describes methods for the production of antibodies, or antibody fragments, which have the same binding specificity as a parent antibody but which have increased human characteristics. Humanized antibodies may be obtained by chain shuffling, perhaps using phage display technology, in as much as such methods will be useful in the present invention the entire text of U.S. Pat. No. 5,565,332 is incorporated herein by reference. Human antibodies may also be produced by transforming B cells with EBV and subsequent cloning of secretors as described by Hoon et al. (1993).

### IV. IDENTIFICATION OF ANTIBODIES TO CYCLIC PEPTIDES

In particular embodiments, the present invention provides a method for identifying an antibody against a protein loop domain. This can be readily determined using any one of variety of immunological screening assays in which antibody competition can be assessed.

Immunoassays, in their most simple and direct sense, are binding assays. Certain preferred immunoassays are the various types of enzyme linked immunosorbent assays (ELISAs), radioimmunoassays (RIA) and immunobead capture assay. Immunohistochemical detection using tissue sections also is particularly useful. However, it will be readily appreciated that detection is not limited to such techniques, and Western blotting, dot blotting, FACS analyses, and the like also may be used in connection with the present invention. The steps of various useful immunodetection methods have been described in the scientific literature, such as, e.g., Doolittle et al., 1999; Gulbis et al., 1993; De Jager et al., 1993; and Nakamura et al., 1987, each incorporated herein by reference.

Thus, it is contemplated that the cyclic or end-to-end cyclized peptides of the present invention may be employed to identify antibodies having reactivity therewith, using a number of immunoassays. For example, where the test antibodies to be examined are obtained from different source animals, or are even of a different isotype, a simple competition assay may be employed in which the control and test antibodies are premixed and then applied to an antigen composition. By “antigen composition” it is meant any composition that contains a cyclic peptide comprising an ECL domain of a transmembrane protein or related antigen as described herein. Thus, protocols based upon ELISAs and Western blotting are suitable for use in such simple competition studies. Such protocols are described below.

In such embodiments, one would pre-mix the control antibodies with varying amounts of the test antibodies (e.g., 1:1, 1:10 and 1:100) for a period of time prior to applying to an antigen composition, such as an antigen-coated well of an ELISA plate or an antigen adsorbed to a membrane (as in dot blots and Western blots). By using species or isotype secondary antibodies one will be able to detect only the bound control antibodies, the binding of which will be reduced by the presence of a test antibody that recognizes the same epitope/antigen.

In conducting an antibody competition study between a control antibody and any test antibody, one may first label the control with a detectable label, such as, e.g., biotin or an enzymatic, radioactive or fluorescent label, to enable subsequent identification. In these cases, one would incubate the labeled control antibodies with the test antibodies to be examined at various ratios (e.g., 1:1, 1:10 and 1:100) and, after a suitable period of time, one would then assay the reactivity of the labeled control antibodies and compare this with a control value in which no potentially competing test antibody was included in the incubation.

The assay may again be any one of a range of immunological assays based upon antibody hybridization, and the control antibodies would be detected by means of detecting their label, e.g., using streptavidin in the case of biotinylated antibodies or by using a chromogenic substrate in connection with an enzymatic label or by simply detecting a radioactive or fluorescent label. An antibody that binds to substantially the same epitope as the control antibodies will be able to effectively compete for binding and thus will significantly reduce control antibody binding, as evidenced by a reduction in bound label. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference.

The reactivity of the labeled control antibodies in the absence of any test antibody would be the control high value. The control low value would be obtained by incubating the labeled antibodies with unlabelled antibodies of the same type, when competition would occur and reduce binding of the labeled antibodies. A significant reduction in labeled antibody reactivity in the presence of a test antibody is indicative of a test antibody that recognizes the same epitope, i.e., one that “cross-reacts” with the labeled antibody. A significant reduction is a reproducible, i.e., consistently observed, reduction in binding.

Antigenic portions and epitopic core regions of proteins may also be predicted with the use of computer programs. Examples include those programs based upon the Jameson-Wolf analysis (Jameson and Wolf, 1988; Wolf et al., 1988), the program PepPlot® (Brutlag et al., 1990; Weinberger et al., 1985), and other new programs for protein tertiary structure prediction (Fetrow and Bryant, 1993). Another commercially available software program capable of carrying out such analyses is MacVector (IBI, New Haven, Conn.).

A. ELISAs

As discussed above, it is contemplated that a cyclic or end-to-end cyclized peptide of the present invention will find utility in ELISAs. ELISAs have certain features in common, such as coating, incubating or binding, washing to remove non-specifically bound species, and detecting the bound immunecomplexes. In one exemplary ELISA, the cyclic or end-to-end cyclized peptide of the invention are immobilized onto a selected surface exhibiting peptide affinity, such as a well in a polystyrene microtiter plate. Then, a test composition suspected of containing the antibody, is added to the wells. After binding and washing to remove non-specifically bound immunocomplexes, the bound antibody may be detected. Detection may be achieved by the addition of a detectable label, such as for example, horseradish peroxidase.

In coating a plate with an antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a peptide to the well, coating with a non-reactive material to reduce background, and washing to remove unbound material, the immobilizing surface is contacted with the control human clinical or biological sample to be tested under conditions effective to allow immunecomplex (antigen/antibody) formation. Detection of the immunecomplex then requires a labeled secondary binding ligand or antibody, or a secondary binding ligand or antibody in conjunction with a labeled tertiary antibody or third binding ligand.

“Under conditions effective to allow immunecomplex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 h, at temperatures preferably on the order of 25° to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immunecomplex with a urease, glucose oxidase, alkaline phosphatase or hydrogen peroxidase-conjugated antibody for a period of time and under conditions that favor the development of further immunecomplex formation (e.g., incubation for 2 h at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation is then achieved by measuring the degree of color generation, e.g., using a visible spectra spectrophotometer.

In other embodiments, solution-phase competition ELISA is also contemplated. Solution phase ELISA involves attachment of a transmembrane protein or end-to-end cyclized peptide corresponding to a transmembrane protein to a bead, for example a magnetic bead. The bead is then incubated with sera from human and animal origin. After a suitable incubation period to allow for specific interactions to occur, the beads are washed. The specific type of antibody is the detected with an antibody indicator conjugate. The beads are washed and sorted. This complex is the read on an appropriate instrument (fluorescent, electroluminescent, spectrophotometer, depending on the conjugating moiety). The level of antibody binding can thus by quantitated and is directly related to the amount of signal present.

B. Western Blotting

The present invention may also employ the use of Western blotting (immunoblotting) to analyze the binding of an antibody preparation to a cyclic or end-to-end cyclized peptide corresponding to an ECL domain of a transmembrane protein in a cell or cell extract. The Western blotting technique is well known to those of skill in the art, see U.S. Pat. No. 4,452,901 incorporated herein by reference and Sambrook et al. (2001). In brief, this technique generally comprises separating proteins in a sample such as a cell or tissue sample by SDS-PAGE gel electrophoresis. In SDS-PAGE proteins are separated on the basis of molecular weight, then are transferred to a suitable solid support, (such as a nitrocellulose filter, a nylon filter, or derivatized nylon filter), followed by incubation of the proteins on the solid support with antibodies that specifically bind to the proteins, for example, an antibody that specifically recognizes cyclic peptide corresponding to an ECL domain of a transmembrane and/or a cyclized peptide corresponding thereto.

### V. EXAMPLES

The following examples are included to demonstrate exemplary embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Experimental Procedures

Peptide synthesis and cyclization. All peptides were synthesized using either N-Fluorenylmethoxycarbonyl (Fmoc) or N-tert-Butoxycarbonyl (Boc) chemistry. NovaSyn TGA pre-loaded resins were used for Fmoc syntheses and pre-loaded Pam resins used for Boc syntheses. All resins and reagents were from Novabiochem (San Diego, Calif.). Ninhydrin-protected cysteine (Nin-Cys) was synthesized according to McCurdy (1989) and was coupled to peptide resins using HoBt/HBTU activation with coupling time <20 min. Following cleavage and purification, Nin-protected peptides in 60% acetonitrile were added to HEPES buffer (200 mM, pH 7.7) for final peptide concentrations of 50-60 μM. 3-mercaptopropiosulfonic acid and tris-carboxymethylphosphine (both from Sigma, St. Louis, Mo.) were added yielding final concentrations of 50 mM and 5 mM respectively. The solution was shaken at room temperature and time points assayed by RP-HPLC.

For preparation of biotinylated peptides, peptides with deprotected thiols were dissolved in guanidine hydrochloride (6 M) to a concentration of 2 mM and added dropwise to a 3 mM solution of PEO-maleimide activated biotin (Pierce, Rockford, Ill.) in 3 M GnHCl/200 mM PIPES (pH 6.7) to a final peptide concentration of 0.2 mM. The reactions were complete within 1 h.

All peptides were purified by high performance liquid chromatography (HPLC) on a Vydac (Hesperia, Calif.) C₁₈ reversed-phase preparatory column then characterized by analytical HPLC and matrix-assisted laser desorption ionization mass spectroscopy (MALDI-MS).

Cell lines, virus, antibodies. Sup-TI, P4R5 and HeLa cells were obtained from Drs. Aiken and Unumatz. Sup-T1 cells are a non-adherent human leukemia T cell line. P4R5 cells are HeLa cells with a stably integrated LTR-LacZ reporter gene cassette expressing CD4, CXCR4 and CCR5. HIV-1 viruses BaL and NL-43 were provided by Dr. Aiken. The following reagents were obtained through the AIDS Research and Reference Reagent Program, Division of AIDS, NIAID, NIH: Cf2Th/syn CCR5 cells from Dr. Tajib Mirzabekov and Dr. Joseph Sodroski (Mirzabekov et al., 1999) and PM1 cells from Dr. Marvin Reitz (Lusso et al., 1995). Cell lines were maintained in Dulbecco's modified Eagle's medium (DMEM, Gibco BRL) supplemented with 10% fetal bovine serum at 37° C. in 5% CO₂. MAb 2D7 (Wu et al., 1997) was purchased from Pharmingen (San Diego, Calif.).

Solution phase selection method. Spleens from naive newborn and 3-4 week old outbred mice and rats were used to construct a phage-displayed ScFv recombinant antibody library (˜2.9×10⁹ members) according to modifications of a previously published protocol (Pope et al., 1996). Antibodies comprising the library are encoded within the Amersham pCANTAB5E phagemid vector. The vector contains an ampicillin-resistance gene to select for bacterial clones that contain the ScFv-encoding phagemid. All ScFv are expressed either as a phage gene 3 fusion protein (for phage display purposes) or as an epitope-tagged ScFv. Rodent ScFv display an epitope (E-tag) recognized by the anti-E MAb (Amersham Biosciences).

For phage selections, the phage antibody library (˜10³ phage particles) was mixed with approximately 100 pmol of biotinylated peptide in the presence of PBS containing 0.05-0.1% Tween 20 and incubated at room temperature for 2-3 h. The mixture was combined with 100 μg of streptavidin magnetic beads (Pierce cat# Z5481) that had been previously blocked for 1 h in PBS containing 0.1% Tween 20 (PBS-T). After a 5 min incubation, a magnet was used to separate phage antibodies bound to beads from unbound phage antibodies in solution. The beads were washed 7 times with PBS-T and 1-3 times with PBS. The PBS was removed and 50-100 μl of 100 mM treithylamine added to the beads to elute phage antibodies bound to biotinylated peptide captured onto streptavidin magnetic beads.

After a 10 min incubation, the 100 mM treithylamine supernatant containing the eluted phage antibodies was removed, neutralized with 0.5 ml volumes of 1M Tris-HCl and used to infect E. coli TG 1 cells. Infected cells were incubated for 1 h at 37° C., plated onto 2×YT agar plates containing 100 μg/ml ampicillin and 2% glucose (2×YTAG) and incubated overnight at 30° C. After incubation, cells were scraped, grown in 10 ml of 2×YTAG broth at 37° C., infected with M13KO7 helper phage and grown overnight at 30° C. in 10 ml of 2×YT medium containing 100 μg of ampicillin and 50 μg of kanamycin per ml of culture medium to produce phage displayed antibodies for a second round of selection. For a second round of selection, ˜1 ml of phage antibodies in culture supernatant was mixed with 100 pmol of biotinylated peptide and Tween 20 (final concentration of 0.05-0.1%) and incubated for 2-3 h at room temperature. Phage antibodies bound to biotinylated peptides were harvested using streptavidin magnetic beads, eluted, used to infect E. Coli TG 1 cells and plated as per the first round selection. In some cases prior to selections, phage antibodies were cross-absorbed with streptavidin magnetic beads, free biotin or the WKGCGKI peptide to remove, respectively, antibodies reactive with biotin/streptavidin or the WKGCGKI amino acid sequence used to form the cyclic CCR5 peptides.

Assay for ScFv reactive with CCR5 on cell surface. E-tagged ScFv reactive with cellular CCR5 were detected using an Applied Biosystems FMAT 8100 plate reader and the anti-E MAb which had been conjugated to FMAT Blue Dye using an Applied Biosystems FMAT Blue Dye conjugation kit (cat# 431053C). A Genetix Qpix Colony-picker was used to pick bacterial colonies, stemming from second round selections, from 2×YT AG agar plates to 384 well microtiter plates that contained 100 μl/well of 2×YT supplemented with ampicillin (100 μg/ml) and 1 mM IPTG (2×YT A1). Microtiter plates were incubated overnight at 30° C. then centrifuged (˜1,000×g, 10 min) to pellet cells. Supernatants were removed and cell pellets resuspended in 40 μl TES [0.2 M Tris-HCI (pH 8.0), 0.5 mM EDTA, 0.5 M sucrose] and 60 μl of TES diluted 1:5 with distilled water to give a final volume of 100 μl. Resuspended cells were placed on ice for 1 h to prepare soluble E-tagged ScFv in periplasmic extract. HeLa and P4R5 cells (2×10⁵ cells per ml PBS) were plated into Applied Biosystems FMAT 384 well microtiter plates at 20 μl/well after which 25 μl of FMAT Blue Dye conjugated anti-E MAb (250 ng/ml PBS-T) and 25 μl of periplasmic extract were added. Plates were incubated for 2 h at room temperature in the dark then read using the FMAT 8100 plate reader. Bacterial clones producing ScFv reactive with CCR5+ P4R5 cells but not with CCR5− HeLa cells were identified and used to produce ScFv on a large scale for further analysis.

ScFv expression and purification. Individual bacterial colonies producing ScFv reactive with CCR5+ P4R5 cells but not with CCR5− HeLa cells were used to inoculate 500-1,000 ml flasks containing 250 ml 2×YT AG broth. Cells were grown overnight at 30° C. with shaking at 100-125 rpm then centrifuged. Pellets were resuspended in 250 ml 2×YT A1 then grown overnight as above. Cells were pelleted, resuspended in 4 ml of 1×TES and 6 ml of ⅕^{th}×TES and placed on ice to produce soluble E-tagged ScFv in periplasmic extract. E-tagged ScFv were affinity-purified using an anti-E tag MAb column and RPAS Purification Module (Amersham cat#171362-01) according to the manufacturer's instructions.

ELISA. For peptide ELISAs, 96-well polystyrene plates were coated with synthetic linear and cyclic peptides (100 ng/well) or with unrelated peptide as a control antigen in Tris-buffered saline (TBS; 50 mM Tris, 150 mM NaCl, pH 7.5) and incubated overnight at 4° C. After washing 4 times with TBS containing 0.1% Tween 20, wells were blocked (5% skim milk powder in TBS) for 30 min at 37° C., then washed 3 times before the addition of serial doubling dilutions of the selected antibody. Bound antibody was detected by incubation with anti-E-tag-horseradish peroxidase conjugate (Amersham Biosciences), substrate addition resulted in color development which was measured at 450 run.

Western blot. Cf2Th/syn CCR5, PM 1 and HeLa cells were washed three times (0.1 M PBS, pH7.0) then resuspended in lysis buffer (50 mM Tris-HCI, pH 7.5, 150 mM NaCl, 1% NP40 containing 100 μg/ml protein inhibitor) before overnight incubation at 4° C. Treated samples were centrifuged at 12,000 RPM for 15 minutes, and the supernatant carefully transferred into a fresh tube for detection. Then to each tube 50 μl of native sample buffer was added then 20 μl loaded onto a 12% SDS-polyacrylamide gel and electrophoresed for 4 h at 65 V. The fractionated extracts were electro-transferred to Hybond ECL nitrocellulose membranes (Amersham Biosciences) which were then blocked for 1 h. ScFv antibody preparations were adjusted to 10 μg/ml in blocking buffer and the membranes probed for 2 h at RT. Anti-E tag HRP conjugate (1:1000; Amersham Biosciences) was added and membranes incubated for a further 1 h. After washing with blocking buffer the specific binding of various ScFv preparations was detected using an enhanced chemilumeniscence kit (Amersham Biosciences).

Flow cytometry. Sup-T 1 and HeLa cells were harvested, washed twice with PBS and prepared at 1×10⁵ cells per sample in flow cytometry tubes. Addition of 1 μg of ScFv per sample was followed by a 2 h incubation on ice. After 2 washes with PBS consisting of centrifugation (2000 rpm, 5 min) and replacement of supernatant, anti-E-tag FITC conjugate was added and samples incubated a further 2 h on ice. Cells were again washed twice with PBS and analyzed immediately using a FACSCalibur instrument and CellQuest software (Becton Dickinson, USA).

Immunofluorescent staining. Cf2Th/syn CCR5 cells (2×10³) were seeded into 8-well chamber slides and cultured overnight. Non-adherent cells were removed by brief rinsing with PBS and adherent cells fixed to slides (2% paraformaldehyde in the PBS, 10 min). Following a gentle wash (PBS), cells were exposed to penetrating buffer (0.01% Triton X-100 in PBS, 5 min). Specimens were incubated in blocking buffer for 2 h which was then replaced with 200 μl PBS containing 10 μg/ml of ScFv or a 1:200 dilution of MAB 2D7 for control. Following a 2 h incubation at RT with the primary reagent the specimens were stained with a 10 μg/ml solution (100 μl) of anti-E tag FITC conjugate prepared at the Molecular Recognition Unit of Vanderbilt University. The slides were mounted with 50% glycerol in PBS and examined immediately under a fluorescence microscope (Nikon, Japan).

HIV-1 infection assay. A quantitative method based on that of Kimpton and Emerman (1992) was developed. P4R5 cells were seeded into 48-well plates at 4×10⁴ cells pei well in 200 μl of medium without selective antibiotics but containing DEAE-dextran (20 μg/ml). Twenty to 24 h later the cells were infected with either HIV-1 BaL (M-tropic) or HIV-1 NL4-3 (T-tropic) at approximately 1000 focus-forming units per well, in the presence of ScFv antibodies (100 μg/ml). Three days after infection the cells were fixed with paraformaldehyde, stained with X-Gal (5-bromo-4-chloro-3-indolyl-R-D-galactopyranoside) and the number of blue nuclei (infected cells) enumerated using an automated optical imager (NIH image system). Results are expressed as number of blue cells per culture well compared to control wells in which virus was incubated either without ScFv or in the presence of the CCR5-specific MAB 2D7.

### Example 2

### Preparation of Cyclic Peptides

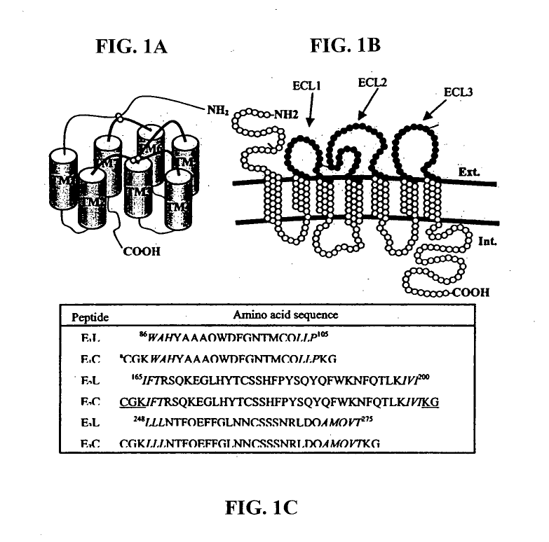

CCR5 consists of seven transmembrane (TM) helical segments which support four extracellular domains, namely the N-terminal tail and three extracellular loops: ECL1, ECL2 and ECL3. The receptor also contains two intra-molecular disulfide bonds, the first located between the N-terminal tail and ECL3 and the second linking ECL1 and ECL2 to create a conformationally constrained molecule (FIG. 1A-1C). In an approach designed to produce peptides that mimic the native structural epitopes within the three loops, end-to-end cyclic peptides were assembled along with linear versions of the extracellular loops. The peptides, termed E₁, E₂ and E₃ linear (L) and cyclic (C), contained the entire loop sequences along with parts of the TM helical bundles that stabilize the ectodomain loops (FIG. 1C). To aid solubility and flexibility in the design of cyclic peptides that require a reverse turn at the shortened TM helical bundle, the residues CGK and KG were included at the N— and C-termini of all peptides. These extra residues also acted as spacers between loop ends in an attempt to create a more natural structure. The Cys residue was included as the sole requirement for end-to-end cyclization using the Cys-thioester method (Zhang and Tam, 1997) to regenerate the thiol after cyclization. This proved to be a critical component of the design strategy as it allowed site-specific conjugation with minimal structural distortion.

Precursors of cyclized peptides were synthesized using conventional solid-phase methodology with Boc chemistry. A novel strategy was utilized for cyclization using ninhydrin-protected Cys (Nin-Cys). Nin-Cys was added to the N-terminal as the final step of solid-phase synthesis. It was found that Nin-Cys could be readily attached to peptide resins during stepwise synthesis with no indication of steric hindrance during the coupling reaction or reactivity at the Nin-Cys thiazolidine ring. The presence of the ninhydrin protects the Cys residue during cleavage with hydrogen fluoride to eliminate disulfide bond formation and simplify purification. The chemoselective ligation was performed in aqueous solution and reached completion within 10 h. This ligation strategy resulted in straightforward purification of linear components by HPLC and a high yield of end-to-end cyclic peptides containing a thiol handle.

Thus, the use of thioester chemistry to yield cyclic peptides with a thiol handle is a significant achievement. Not only does this ligation strategy simplify production of end-to-end cyclic peptides, but it also provides a mechanism by which peptides can be conjugated to numerous reagents for various purposes. Conjugation through a single disulfide bond to fluorescent tags, enzymes, substrates, proteins and reagents such as biotin will prove to be useful in situations where a chemically defined molecule is required. A disulfide bond may be easily reduced to release the peptide, a characteristic especially useful for panning and selection strategies. Furthermore, the simplicity and flexibility of the ligation and conjugation reactions (Tam et al., 2001) allows for the employment of the strategies without extensive knowledge of peptide chemistry or dedicated equipment.

### Example 3

### Generating Antibody Libraries

Genes encoding heavy and light chain variable antibody fragments [Fv(s)], were obtained from human or rodent B-lymphocytes. The Fv antibody genes were used to generate the antibody libraries. Heavy and light chain Fv DNA, randomly assembled into singe chain Fv (ScFv) encodes for the antigen-binding domain of an antibody. The ScFv DNA was ligated to a phagemid vector, which was then used to transform E. coli. Transformed E. coli was subsequently rescued with helper phage (M13K07) to establish an active phage (bacterial virus) infection. The infected E. coli secreted newly formed phage particles that contained the ScFv-phagemid DNA and expressed or displayed the ScFv antibodies on the phage tip. The phage-displayed human and rodent recombinant ScFv antibody libraries contain, respectively, 4-60 billion or 2+ billion different antibodies. Each ScFv antibody has a different amino acid sequence. Since the ScFv's amino acid sequence determines its antigen specificity, the libraries theoretically contain antibodies to a billion or more different antigenic sites.

ScFv antibodies specific for antigens can be selected from the phage libraries within a few weeks without the need for animal immunizations. Each library contains a small number of phage, which display an antibody specific for an antigen. Phage displaying antibodies specific for an antigen (e.g., a peptide, protein, hapten, etc.) are selected by panning the phage libraries against immobilized or biotinylated antigen. Non-specific phage antibodies are washed away from the antigen. Bound phage are chemically or biologically stripped from the antigen and used to infect E. coli. The infected E. coli are plated out onto the agar surface of a petri dish, and allowed to grow to produce colonies. Each colony arising from a phage-infected E. coli bacterium will contain approximately 100 million bacteria, and each bacterium in the colony contains a copy of the antibody gene. All of the colonies on the petri dish will be rescued with helper phage to establish another phage infection. Each of the 100 million or so bacteria present in a colony (rescued with helper phage) will produce 100 million or more copies of the antigen-specific phage-displayed antibody. The number of antigen-specific phage-displayed antibodies, which was relatively small in the original library, has increased a thousand to a million fold after the first round of antigen selection and helper phage rescue. Subsequent rounds of antigen selection and helper phage rescue are used to enrich for and obtain the most desirable antigen-specific antibodies. The final phage-infected E. coli can be then be used to express the ScFv recombinant antibody by itself. Antigen-specific ScFv produced by each E. coli clone is monoclonal, and can be used in various immunoassays to detect or quantitate an antigen.

### Example 4

### Selection of Antigen-Specific Antibodies using Phage-Displayed Antibody Libraries

DNA microarrays can be used to identify cDNAs that encode for new or novel proteins. Cyclic or constrained peptides, which represent domains on these proteins, can be used as antigens to obtain antibodies specific to the proteins. Large human or rodent phage-displayed recombinant antibody libraries can be applied to immobilized or biotinylated peptides for a first round of antigen selection. Unbound non-specific phage antibodies are washed away, and the bound phage is chemically or biologically stripped from the antigen. Phage-displayed antibodies, stripped from the peptides, can be used to infect E. coli. The infected E. coli is plated out onto the agar surface of a petri dish, and allowed to grow to produce colonies. Colonies, scrapped from the surface of the agar, are helper phage rescued to produce phage-displayed antibodies. The newly formed phage, which display an enriched population of antibodies that interacted with the antigen during the first round of selection, are again applied to the peptides for a second round of selection. Bacterial colonies obtained from the second round (or if need be, third or fourth rounds) of antigen selection are picked and transferred to 96 well microtiter plates which contain liquid bacterial culture medium. The bacterial clones are grown and induced to express soluble antibodies. ELISA assays the antibodies against positive and negative control peptides to identify bacterial clones that produce antigen specific antibodies. Larger amounts of antibodies produced by positive bacterial clones can be produced and used to detect novel protein antigens in a variety of assays (e.g., Western blots, immunoprecipitations and immunohistochemistry).

### Example 5

### ScFv Selection Strategy

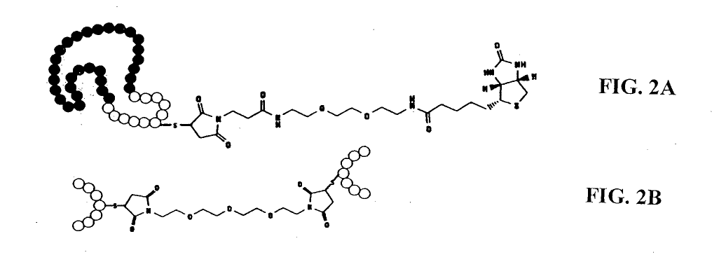

A solution-phase phage selection method was used to preserve the conformational integrity of the linear and cyclic peptides. Instead of using the standard approach with peptides coated directly onto beads before addition of phage, phages were exposed to peptides in solution. The development of the solution-phase method was necessitated by earlier results where single chain antibodies selected using peptide antigens coated directly onto beads displayed a high level of cross-reactivity with non-homologous peptide antigens (data not shown). Because the antigen design included parts of the TM sequence and non-CCR5 residues added to the termini to aid cyclization, these antigens likely shared only these highly hydrophobic residues. For the solution-phase method, biotin was attached via a linker either to the additional Cys of linear peptides or to the thiol handle resulting from peptide cyclization to afford biotinylated-peptides (b-peptides, FIG. 2A). The b-peptides were incubated with phage that had been pre-incubated with a linker peptide designed to remove phages specific for the linker and additional residues (FIG. 2B). To allow washing and phage elution, the high affinity biotin-streptavidin interaction was exploited with the b-peptide-phage assemblies captured by streptavidin-coated beads. Only peptide-bound phage were retained whereas low affinity binders or those specific for either the linker or the additional residues were removed. The peptide-specific phage were eluted during washing with peptide. Phage were selected by this solution-phase method for binding to E1L, E1C, E2L, E2C, E3L, and E3C.

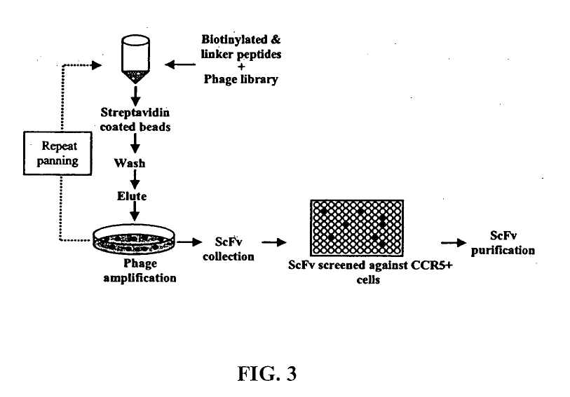

To further increase the probability of selecting biologically active single chain antibodies (ScFv antibodies), a number of peptide-specific phages were cultured and ScFv antibodies collected and tested for binding to CCR5+ cells. For this, an FMAT 8100 system (PE Biosystems, CA) was utilized with aliquots of CCR5+ and CCR5− cells allowed to settle in 384-well plates. After addition of ScFv antibodies and anti-ScFv fluorescein conjugate the fluorescence signal recorded at the extreme bottom of each well was used to determine which antibodies bound specifically to CCR5+ cells. ScFv antibody preparations that exhibited strong binding to cells were purified. The entire selection procedure is outlined in FIG. 3.

### Example 6

### Accessibility of Scfv Epitopes in Solid Phase Peptides

Following phage selection and ScFv antibody purification, antibody specificity was determined by ELISA. The purified ScFv antibody preparations were tested for binding to linear and cyclic peptides coated directly onto plastic wells. Each preparation was tested for binding to both the linear and cyclic forms, regardless of the peptide used for initial phage selection. The ELISA results are summarized in Table 2. In some cases limited quantities of ScFv antibodies precluded use in ELISA. Antibodies fell into four general categories, those that bound a) both linear and cyclic forms, b) linear but not cyclic, c) cyclic but not linear and d) neither linear nor cyclic. Given that the antibodies were from phages originally selected for binding to this same set of peptides, in some cases the ELISA results seem to conflict with the selection procedure. For example, ScFv antibodies H2 and K1 which were selected for binding to E₁C would be expected to bind to E₁C in ELISA and may also bind to E₁L. However, no recognition of E₁C was observed in ELISA. In another example, ScFv antibody O17 was from a phage that bound to E₃C yet the antibody bound to neither E₃C nor E₃L in ELISA. These ambiguous results can be attributed to differences in epitope exposure within solution-phase peptides versus solid-phase peptides and imply that this standard ELISA method is unsuitable for determination of antibody binding capacity to structural epitopes, thus no further analysis of ELISA results was performed. This result highlights the challenge associated with analysis of antibodies specific for structural epitopes.

TABLE 2
Characterization of single chain antibodies directed to extracellular domains of CCR5

| | | | | | Flow cytometry | | | |
|---|---|---|---|---|---|---|---|---|
| Phage- | | | | | Increased | Increased no. | | |
| screening | ScFv | ELISA | | Western | mean cell | of fluorescent | Immuno- | HIV |
| peptide | name | Linear | Cyclic | blot | fluorescence | cells | fluorescence | Inhibition |
| E₁L | G4 | +^{a} | −^{b} | − | − | + | − | − |
| | M5 | − | ND^{c} | − | − | + | − | − |
| | N4 | + | + | − | + | − | − | − |
| E₁C | A1 | + | + | + | + | − | + | + |
| | H2 | + | − | − | − | + | − | − |
| | K1 | + | − | − | − | + | + | − |
| E₂L | B22 | + | ND | − | ND | ND | − | − |
| | C20 | + | ND | − | ND | ND | − | − |
| | K21 | + | ND | − | ND | ND | − | − |
| | P21 | + | + | + | − | + | − | − |
| E₂C | B7 | − | − | − | + | + | + | + |
| | F12 | + | + | − | + | + | − | − |
| | G11 | + | − | + | − | + | − | − |
| | K7 | + | − | − | ND | ND | − | − |
| | K8 | + | + | − | + | + | − | − |
| | L9 | + | + | − | − | + | − | + |
| E₃L | M12 | − | − | − | ND | ND | − | − |
| E₃C | N18 | − | + | + | − | + | − | − |
| | O17 | − | − | + | − | + | − | − |

^{a}+ indicates higher signal compared to control
^{b}− indicates no binding or negative result in test
^{c}ND = Not Determined. Limiting quantity of antibody precluded use

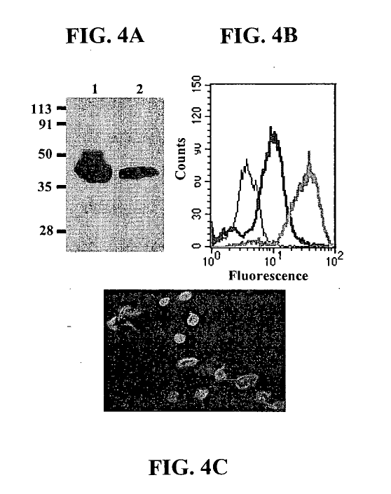

The ScFv antibodies were next tested by Western blot for binding to CCR5 expressed by Cf2Th/syn CCR5 and PM 1 cells. As a negative control, ScFv antibodies were tested for binding to lysates of HeLa cells, a cell line that does not express CCR5. In these cases, no binding was observed. Five ScFv antibodies exhibited binding to denatured CCR5 of both cell lines: A1, P21, G11, N18 and O17 (FIG. 4A; Table 2). One ScFv antibody, P21, was from a phage that bound a linear peptide, the remaining antibodies were from phages selected by binding to cyclic peptides. It is not surprising that only a small number of antibodies were found to bind to the denatured CCR5 given that the selection strategy was designed to favor antibodies specific for structural epitopes.

The conventional phage selection protocol was altered to include a second solution-phase step in order to preserve peptide structure. The inconclusive ELISA results subsequently showed that the peptides did in fact differ in structure in solution and on solid phase. The use of the solution-phase selection step aided in the identification of relevant antibodies with specificity for CCR5 in their normal physiological state. Reliance on peptide ELISA for selection of potentially active antibodies would have resulted in at least one of the active antibodies being excluded from further tests. The results indicate that this two-step high throughput method can be routinely applied to ScFv antibody selection.

### Example 7

### Scfv Antibodies Bind to Native CCR5

To confirm the viability of the selection method and to further characterize the selected antibodies a flow cytometry protocol was devised to mimic the FMAT selection process. As expected, after incubation of CCR5+ Sup-T1 cells with ScFv antibodies and subsequent staining with anti-E tag FITC conjugate, it was found that antibody presence increased cellular fluorescence when compared to samples which did not receive ScFv antibody. Antibodies did not bind to the CCR5− HeLa cells. On closer examination of the data, ScFv antibodies were observed to produce three different binding profiles. Of the 14 antibodies tested (limited quantities of the remainder prevented use), induced an increase in the number of fluorescent cells but not fluorescence intensity per cell. The epitopes recognized by these antibodies occur with low frequency on the cell surface leading to a low concentration of antibody per cell. Two antibodies (N4 and A1; FIG. 4B) induced an increase in fluorescent intensity but not in the number of fluorescent cells. In these cases the antibodies bound to epitopes that exist at a relatively high density on cells but only on a limited portion of cells. The remaining 3 antibodies (B7, F12 and K8) induced both elevated fluorescent intensity per cell and number of fluorescent cells. This binding pattern indicates that the epitopes occur at high frequency on large number of cells. The results are summarized in Table 2. This data supports previous reports of CCR5 existing in several conformations on cell surface (Lee et al., 1999; Cornier et al., 2000; Farzan et al., 1998; Benkirane et al., 1997; Xiao et al., 1999) and that antibodies are specific for various epitopes within CCR5. Correlation between binding profile and ability of antibodies to inhibit HIV infection is discussed in proceeding sections.

While all ScFv antibodies were detected to bind to CCR5+ cells by flow cytometry, only three bound to Cf2Th/syn CCR5 cells with adequate affinity to allow visualization by fluorescence microscopy: A1, B7 and K1 (FIG. 4C). All three were selected on cyclic peptides and displayed different binding profiles in flow cytometric analysis.

### Example 8

### Prevention of HIV Infection of CCR5+ Cells

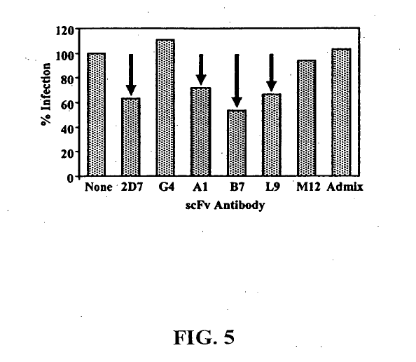

Of the 19 ScFv antibodies tested in a single-cycle infectivity assay, three partially inhibited infection of P4R5 cells with M-tropic HIV-1 BaL (ScFv A1, B7 and L9; FIG. 5, Table 2), confirming the hypothesis that peptides representing structural epitopes of the CCR5 ectodomains would be effective antigens. A mixture of three ScFv antibodies, N4, P21 and O17 which were specific for ECL1, ECL2 and ECL3 respectively, did not prevent viral infection even at high concentration (100 μg/ml). The active antibodies were not able to inhibit infection with T-tropic HIV-1 NL4-3 which uses CXCR4 as the coreceptor. The inhibition of infection induced by the ScFv antibodies A1, B7 and L9 was approximately equal to, if not better than, that of MAB 2D7.

Thus, this study show that three ScFv antibodies selected by structural epitopes of the CCR5 ectodomains are as effective as the MAB 2D7 in preventing HIV infection in vitro. These ScFv antibodies are specific and do not inhibit infection with T-tropic HIV-1 NL4-3 which uses CXCR4 as the coreceptor.

### Example 9

### Correlation of Antibody Binding Profiles HIV Inhibitory Activity

A number of antibodies that displayed strong binding to denatured and even native protein in Western blots and/or immunofluorescence studies were incapable of preventing HIV infection of target cells. These antibodies, K1, P21, G11, N18 and O17, have proven to bind strongly to CCR5 but it seems that each lacks specificity for residues critical to HIV entry. To further streamline the selection process described here it, may be necessary to identify and remove such antibodies prior to testing for viral inhibition.

The three ScFv antibodies that did inhibit HIV infection were all selected for binding to cyclic peptides, one against E₁C and two against E₂C. Characterization by ELISA, Western blot and flow cytometry did not reveal any further similarities between the antibodies. A1 antibody was initially selected for binding to E₁C. This antibody bound to E₁C and E₁L solid-phase peptides, denatured protein, CCR5+ cells in flow cytometry and immunofluorescence and prevented infection. Antibody B7, selected for binding to E₂C, possessed a different profile from A1. This antibody did not recognize epitopes in ELISA nor Western blot, yet exhibited pronounced binding to native CCR5 in flow cytometry and immunofluorescent assays. Finally, ScFv L9 which was also selected due to affinity for E₂C, differed to B7 in that binding to solid-phase peptides in ELISA was observed but visualization by immunofluorescence was absent.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,452,901
U.S. Pat. No. 4,554,101
U.S. Pat. No. 4,816,567
U.S. Pat. No. 4,867,973
U.S. Pat. No. 5,322,783
U.S. Pat. No. 5,384,253
U.S. Pat. No. 5,538,877
U.S. Pat. No. 5,538,880
U.S. Pat. No. 5,550,318
U.S. Pat. No. 5,563,055
U.S. Pat. No. 5,565,332
U.S. Pat. No. 5,580,859
U.S. Pat. No. 5,589,466
U.S. Pat. No. 5,609,870
U.S. Pat. No. 5,610,042
U.S. Pat. No. 5,656,610
U.S. Pat. No. 5,693,762
U.S. Pat. No. 5,702,932
U.S. Pat. No. 5,736,524
U.S. Pat. No. 5,780,448
U.S. Pat. No. 5,785,970
U.S. Pat. No. 5,789,215
U.S. Pat. No. 5,945,100
U.S. Pat. No. 5,980,912
U.S. Pat. No. 5,981,274
U.S. Pat. No. 5,994,624
U.S. Pat. No. 6,027,727
Abbondanzo et al., Breast Cancer Res. Treat., 16:182(151), 1990.
Ali et al., FEBS Lett., 459:230-232, 1999.
Allred et al., Breast Cancer Res. Treat., 16:182(149), 1990.
Barany and Merrifield, In: The Peptides, Gross and Meienhofer (Eds.), Academic Press, NY, 1-284, 1979.
Benjamini et al., Adv. Exp. Med. Biol., 303:71-77, 1991.
Benkirane et al., J. Biol. Chem., 272:30603-30606, 1997.
Blanpain et al., Mol. Biol. Cell, 13:723-737, 2002.
Bouhlal et al., J. Immunol., 166:7606-7611, 2001.
Brown et al. Immunol. Ser., 53:69-82, 1990.
Brutlag et al., Comput. Appl. Biosci., 6(3):237-245, 1990.
Cairns and D'Souza, Nat. Med., 4:563-568, 1998.
Campbell, In: Monoclonal Antibody Technology, Laboratory Techniques in Biochemistry and Molecular Biology, Burden and Von Knippenberg (Eds.), Elseview, Amsterdam, 13: 75-83, 1984.
Capaldi et al., Biochem. Biophys. Res. Comm., 74(2):425-433, 1977.
Chackerian et al., Proc. Nad. Acad. Sci. USA, 96:2373-2378, 1999.
Chou and Fasman, Adv. Enzymol., 47:45-148, 1978a.
Chou and Fasman, Annu. Rev. Biochem., 47:251-276, 1978b.
Chou and Fasman, Biochemistry, 13(2):211-222, 1974a.
Chou and Fasman, Biochemistry, 13(2):222-245, 1974b.
Chou and Fasman, Biophys. J., 26(3):385-399, 1979.
Connor et al., J. Exp. Med., 185:621-628, 1997.
Cornier et al., Proc. Natl. Acad. Sci. USA, 97:5762-5767, 2000.
De Jager et al., Semin. Nucl. Med., 23(2):165-179, 1993.
Defoort et al., Proc. Nad. Acad. Sci. USA, 89:879-3883, 1992.
Deng et al., Nature, 381:661-666, 1996.
Doolittle and Ben-Zeev, Methods Mol Biol, 109:215-237, 1999.
Drin et al., AAPS PharmSci., 4(4):26, 2002.
Farzan et al., J. Virol., 72:1160-1164, 1998
Fetrow and Bryant, Biotechnology (NY), 11(4):479-484, 1993.
Gefter, et al., Somatic Cell Genet., 3:231-236, 1977.
Goding, In: Monoclonal Antibodies: Principles and Practice, 2d ed., Academic Press, Orlando, Fla., pp 60-61, 71-74, 1986.
Goding, In: Monoclonal Antibodies: Principles and Practice, 2d ed., Academic Press, Orlando, Fla., pp 65-66, 1986.
Griffith, EMBO J, 13:3245-3260, 1994.
Gulbis and Galand, Hum. Pathol., 24(12):1271-1285, 1993.
Harland and Weintraub, J. Cell Biol., 101(3):1094-1099, 1985.
Harlow and Lane, In: Antibodies: A Laboratory Manual, Cold Spring Harbor Laboratory, Cold Spring Harbor, N.Y., 346-348, 1988.
Hoon et al., J. Urol., 150(6):2013-2018, 1993.
Huang et al., Nat. Med., 2:1240-1243, 1996.
Jameson and Wolf, Comput. Appl. Biosci., 4(1):181-186, 1988.
Johnson et al., In: Biotechnology And Pharmacy, Pezzuto et al. (Eds.), Chapman and Hall, NY, 1993.
Kazmierski et al., Bioorg. Med. Chem., 11:2663-2676, 2003.
Kimpton and Emerman, J. Virol., 66:2232-2239, 1992.
Kohler and Milstein, Eur. J. Immunol., 6:511-519, 1976.
Kohler and Milstein, Nature, 256:495-497, 1975.
Kyte and Doolittle, J. Mol. Biol., 157(1):105-132, 1982.
Lee et al., J. Biol. Chem., 274:9617-9626, 1999.
Lehner et al., Eur. J. Immunol., 29:2427-2435, 1999.
Lehner et al., J. Immunol., 166:7446-7455, 2001.
Liu et al., Cell, 86:367-377, 1996.
Lopalco et al., J. Immunol., 164:3426-3433, 2000.
Lorens et al., Mol. Ther., 1(5Pt1):438-437, 2000.
Lowman et al., In: Materials for controlled release applications, McCulloch and Shalaby (Eds.), American Chemical Society, 156-164, Washington D.C., 1998.
Lusso et al., J. Virol., 69:3712-3720, 1995.
McCurdy, Pept. Res., 2:47-152, 1989.
McMichael and Hanke, Nat. Med., 9:874-880, 2003.
Merrifield, Science, 232(4748):341-347, 1986.
Mirzabekov et al., J. Biol. Chem., 274:28745-28750, 1999.
Moore et al., Curr. Opin. Immunol., 9:551-562, 1997.
Mora and Tam, J. Immunol., 161:3616-3623, 1998.
Nakamura et al., In: Handbook of Experimental Immunology (4^{th} Ed.), Weir et al. (Eds.), 1:27, Blackwell Scientific Publ., Oxford, 1987.
Olson et al., J. Virol., 73:4145-4155, 1999.
Osbourn et al., Nat. Biotechnol., 16:778-781, 1998.
Paxton et al., AIDS Res. Hum. Retroviruses, 12, 1203-1207, 1996.
Paxton et al., AIDS Res. Hum. Retroviruses, 14(1):S89-S92, 1998.
PCT Appln. WO 93/06213
PCT Appln. WO 94/09699
PCT Appln. WO 95/06128
Pope et al., In: Antibody Engineering: A Practical Approach, IRL Press, New York, 1996.
Potter et al., Proc. Natl. Acad. Sci. USA, 81:7161-7165, 1984.
Quillent et al., Lancet., 351:4-18, 1998.
Sambrook et al., In: Molecular cloning, Cold Spring Harbor Laboratory Press, Cold Spring Harbor, N.Y., 2001.
Steinberger et al., J. Biol. Chem., 275:36073-36078, 2000.
Stewart and Young, In: Solid Phase Peptide Synthesis, 2d. ed., Pierce Chemical Co., 1984.
Tam et al., Biopolymers, 60:194-205, 2001.
Tam et al., J. Am. Chem. Soc., 105:6442, 1983.
Tam, Proc. Natl. Acad. Sci. USA, 85:5409-5413, 1988.
Trkola et al., J. Virol., 75:579-588, 2001.
Tsamis et al., J. Virol., 77:5201-5208, 2003.
Tur-Kaspa et al., Mol. Cell Biol., 6:716-718, 1986.
Veljkovic et al., Vaccine, 19:1855-1862, 2001.
Wada et al., Nucleic Acids Res. 18:2367-2411, 1990.
Wang et al., Nat. Med., 5:1004-1009, 1999.
Waterhouse et al., Nucl. Acids Res., 21:2265-2266, 1993.
Weinberger et al., Science, 228:740-742, 1985.
Wolf et al., Comput. Appl. Biosci., 4(1):187-191, 1988.
Wuetal., J. Exp. Med., 185:1681-1691, 1997.
Xiao et al., Proc. Natl. Acad. Sci. USA, 96:7496-7501, 1999.
Zhang and Tam, J. Am. Chem. Soc., 119:2363-2370, 1997.
Zuber et al., Virology, 278:400-411, 2000.

What is claimed:

1. A method for preparing an antibody against a protein loop domain comprising:
(a) providing a cyclic peptide corresponding to said protein loop domain;
(b) administering said cyclic peptide to an experimental animal under conditions supporting production of antibodies; and
(c) obtaining from said animal (i) an antibody that binds to said protein loop domain or (ii) an antibody-producing cell, antibodies from which bind to said protein loop domain.

2. The method of claim 1, wherein said protein is transmembrane protein.

3. The method of claim 2, wherein said transmembrane protein is a G-protein coupled receptor.

4. The method of claim 2, wherein said transmembrane protein is a viral envelope protein.

5. The method of claim 1, wherein said experimental animal is a mouse, a rat, a rabbit, a guinea pig, a goat, a sheep, a non-human primate or a human.

6. The method of claim 1, wherein step (c) comprises obtaining an antibody.

7. The method of claim 1, wherein step (c) comprises obtaining an antibody-producing cell.

8. The method of claim 7, further comprising the steps of fusing an antibody-producing cell with an immortalized cell.

9. The method of claim 1, further comprising assessing binding of said antibody with a corresponding native protein.

10. The method of claim 1, further comprising assessing binding of said antibody with said cyclic peptide.

11. The method of claim 1, wherein said cyclic peptide is linked to a carrier molecule.

12. The method of claim 1, wherein said cyclic peptide of step (b) is mixed with an adjuvant.

13. A method for identifying an antibody against a loop domain of a protein comprising:
(a) providing a cyclic peptide corresponding to said loop domain;
(b) contacting said cyclic peptide with a population of antibodies; and
(c) identifying an antibody that binds to said loop domain.

14. The method of claim 13, wherein said protein is a transmembrane protein.

15. The method of claim 13, wherein said experimental animal is a mouse, a rat, a rabbit, a guinea pig, a goat, a sheep, a non-human primate or a human.

16. The method of claim 13, wherein said transmembrane protein is a viral envelope protein or a G-protein coupled receptor.

17. The method of claim 16, wherein said G-protein coupled receptor is CCR5, CXCR4, CR7 and CXCR3.

18. The method of claim 16, wherein said viral envelope protein is a human rhinovirus protein, influenza A virus protein, sendai virus protein, Herpes simplex virus (type 1) protein, Epstein-Barr virus protein, vesicular stomatitis virus protein, rabies virus protein, a simian immunodeficiency virus protein, or a human immunodeficiency virus protein.

19. The method of claim 13, wherein said population of antibodies is expressed on the surface of a phage library.

20. The method of claim 13, wherein said population of antibodies is comprised in an antibody array.

21. The method of claim 13, further comprising assessing binding of said antibody with a corresponding native protein.

22. The method of claim 13, further comprising assessing binding of said antibody with said cyclic peptide.

23. The method of claim 20, wherein said peptide is labeled and mixed in solution with said phage library.

24. An end-to-end cyclized peptide corresponding to a loop domain of a protein.

25. The peptide of claim 24, wherein the loop domain is from a transmembrane protein.

26. The peptide of claim 25, wherein the transmembrane protein loop domain is an extracellular loop domain.

27. The peptide of claim 25, wherein the transmembrane protein loop domain is an intracellular loop domain.

28. The peptide of claim 24, further comprising a heterologous thiol residue.

29. The peptide of claim 24, wherein said peptide is labeled.

30. The peptide of claim 24, wherein said label is a colorimetric label, a chemilluminescent label, a fluorimetric label, a radiolabel, a dye, or a ligand.

31. The peptide of claim 24, wherein said transmembrane protein is G-protein coupled receptor.

32. The peptide of claim 31, wherein said G-protein coupled receptor is CCR5, CXCR4, CR7 and CXCR3.

33. The peptide of claim 24, wherein said cyclized peptide comprises an amide bond that fuses both ends.

34. The peptide of claim 24, wherein said transmembrane protein is a viral envelope protein.

35. The peptide of claim 34, wherein said viral envelope protein is a human rhinovirus protein, influenza A virus protein, sendai virus protein, Herpes simplex virus (type 1) protein, Epstein-Barr virus protein, vesicular stomatitis virus protein, rabies virus protein, a simian immunodeficiency virus protein, or a human immunodeficiency virus protein.

36. The peptide of claim 24, wherein said peptide corresponds to a plurality of loop domains.

37. The peptide of claim 36, wherein said plurality of loop domains comprises more than one extracellular loop domain of a transmembrane protein.

38. The peptide of claim 36, wherein said plurality of loop domains comprises more than one intracelluar loop domain of a transmembrane protein.

39. The peptide of claim 36, wherein said plurality of loop domains comprises at least one extracellular loop domain and at least one intracellular loop domain of a transmembrane protein.

40. A composition comprising a plurality of end-to-end cyclized peptides corresponding to a plurality of loop domains of a protein.


SEQUENCE LISTING (6 sequences)

SEQ ID NO: 1 - protein, 20 aa, Artificial Sequence
  Description of Artificial Sequence  Synthetic Peptide
WAHYAAAQWDFGNTMCQLLP

SEQ ID NO: 2 - protein, 25 aa, Artificial Sequence
  Description of Artificial Sequence  Synthetic Peptide
CGKWAHYAAAQWDFGNTMCQLLPKG

SEQ ID NO: 3 - protein, 35 aa, Artificial Sequence
  Description of Artificial Sequence  Synthetic Peptide
IFTRSQKEGLHYTCSSHFPYSQYQFWKNFQTKIVI

SEQ ID NO: 4 - protein, 41 aa, Artificial Sequence
  Description of Artificial Sequence  Synthetic Peptide
CGKIFTRSQKEGLHYTCSSHFPYSQYQFWKNFQTLKIVIKG

SEQ ID NO: 5 - protein, 28 aa, Artificial Sequence
  Description of Artificial Sequence  Synthetic Peptide
LLLNTFQEFFGLNNCSSSNRLDQAMQVT

SEQ ID NO: 6 - protein, 33 aa, Artificial Sequence
  Description of Artificial Sequence  Synthetic Peptide
CGKLLLNTFQEFFGLNNCSSSNRLDQAMQVTKG